(19) United States
(12) Patent Application Publication
(10) Pub. No.: US 2015/0212099 A1
(43) Pub. Date: Jul. 30, 2015

(54) METHODS AND COMPOSITIONS FOR ASSAYING VITAMIN D

(71) Applicant: General Atomics, San Diego, CA (US)

(72) Inventors: Chong-Sheng YUAN, San Diego, CA (US); Fakhri Ben Habib SAIDA, San Diego, CA (US); Xiaoru CHEN, San Diego, CA (US); Chao DOU, San Diego, CA (US)

(21) Appl. No.: 14/169,118

(22) Filed: Jan. 30, 2014

(51) Int. Cl. G01N 33/82

(52) U.S. Cl. CPC G01N 33/82; G01N 2333/575

(57) ABSTRACT

This invention generally relates to the field of vitamin D detection. In particular, the invention provides novel methods and kits for assaying a vitamin D moiety in a sample such as a biological fluid.

### FIELD OF THE INVENTION

This invention generally relates to the field of vitamin D detection. In particular, the invention provides novel methods and kits for assaying a vitamin D moiety in a sample such as a biological fluid.

### BACKGROUND OF THE INVENTION

Vitamin D is a steroid-like, fat soluble prohormone. Vitamin D has two main forms: D₂ (ergocalciferol) and D₃ (cholecalciferol). Vitamin D₃ can be manufactured by the body upon exposure to UV radiation. Both Vitamin D₃ and Vitamin D₂ are converted to the active hormone 1,25-dihydroxy Vitamin D through their metabolism in the liver and kidney.

Vitamin D₃ is synthesized in skin by exposure to sunlight (ultraviolet radiation) and obtained from the diet primarily from fish liver oils and egg yolks. Vitamin D₂ is obtained mainly from nutritional supplements and the only prescription drug for Vitamin D deficiency is made of Vitamin D₂. Vitamin D₃ or D₂ is metabolized by the liver to 25(OH)D, which is then converted by the kidneys to 1,25(OH)2D. 25(OH) Vitamin D is the major circulating form which reflects the levels of Vitamin D in the body, but 1,25(OH)2 Vitamin D is the most biologically active form.

Inadequate exposure to sunlight or low intake from diet or supplements may cause vitamin D deficiency. Vitamin D deficiency impairs bone mineralization, causing rickets in children and osteomalacia in adults and may contribute to osteoporosis. Recent studies have shown that Vitamin D deficiency is also linked to cancers, cardiovascular diseases, diabetes, multiple sclerosis, Parkinson disease, Alzheimer's disease, drug efficacy, and all-cause mortality.

A typical normal or sufficient range for Vitamin D is about 30-100 ng/mL. Vitamin D level at about 10-30 ng/mL is considered deficient. Vitamin D level less than 10 ng/mL is considered severely deficient. Vitamin D level more than 150 ng/mL is considered toxic.

Various vitamin D assays are known in the art. For example, Various vitamin D assays are disclosed in U.S. Pat. Nos. 5,821,020, 7,087,395 B1, 7,482,162 B2, 7,964,363 B2, 8,133,694 B2, U.S. patent publication No. 2004/0132104 A1 and WO 2012/091569 A1. At the present time, all the known commercially available vitamin D assays are heterogeneous assays in format that requires phase separation steps (washing steps), which are time consuming and require special instruments such as chemiluminescence immunoassay analyzers, HPLC, or LC-MS instruments.

There remains a need for a robust but cost-effective method for assaying a vitamin D moiety in a sample such as a biological fluid, particularly a method that is a homogeneous assay in format that is applicable to routinely used clinical chemistry analyzers available in all sizes of clinical laboratory settings. The present invention addresses this and other related needs in the field.

### BRIEF SUMMARY OF THE INVENTION

In one aspect, the present disclosure provides for a method for assaying a vitamin D moiety in a sample, which method comprises: a) contacting a sample containing or suspected of containing a vitamin D moiety with 1) a buffer of acidic pH, 2) a specific binding partner that specifically binds to said vitamin D moiety, if present in said sample, to form a vitamin D moiety/specific binding partner complex, said binding partner being different from a natural vitamin D binding protein for said vitamin D moiety, 3) an enzyme donor (ED)-vitamin D moiety conjugate, said enzyme donor (ED) comprising a first fragment of a β-galactosidase, and 4) an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase, said sample is contacted with said buffer of acidic pH and said specific binding partner in one or more steps, and said sample is contacted with said ED-vitamin D moiety conjugate and said enzyme acceptor (EA) in other separate one or more steps; and b) assessing binding between said specific binding partner and said vitamin D moiety to determine the presence, absence and/or amount of said vitamin D moiety in said sample by measuring the activity of said reassembled active β-galactosidase in the presence of a β-galactosidase substrate. Preferably, the above method utilizes the cloned enzyme donor immunoassay (CEDIA) principle.

In another aspect, the present disclosure provides for a kit for assaying a vitamin D moiety in a sample, which kit comprises: a) a buffer of acidic pH, b) a specific binding partner that specifically binds to a vitamin D moiety, if present in said sample, to form a vitamin D moiety/specific binding partner complex, said binding partner being different from a natural vitamin D binding protein for said vitamin D moiety, c) an enzyme donor (ED)-vitamin D moiety conjugate, said enzyme donor (ED) comprising a first fragment of a β-galactosidase, and d) an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

In still another aspect, the present disclosure provides for a reaction mixture for assaying a vitamin D moiety in a sample, which reaction mixture comprises: a) a specific binding partner that specifically binds to a vitamin D moiety, if present in said sample, to form a vitamin D moiety/specific binding partner complex, said binding partner being different from a natural vitamin D binding protein for said vitamin D moiety, b) an enzyme donor (ED)-vitamin D moiety conjugate, said enzyme donor (ED) comprising a first fragment of a β-galactosidase, and c) an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase. In some embodiments, the reaction mixture exists in an acidic environment.

### BRIEF DESCRIPTION OF THE DRAWINGS

FIG. 1 illustrates an exemplary 25(OH)D homogeneous enzyme immunoassay calibration curve on Roche Modular P.

FIG. 2 illustrates an exemplary total 25(OH)D homogeneous enzyme immunoassay method comparison (Roche Modular P) with Diasorin Liason method.

FIG. 3 illustrates an exemplary total 25(OH)D homogeneous enzyme immunoassay linearity on Roche Modular P.

FIG. 4 illustrates an exemplary 25(OH)D homogeneous enzyme immunoassay calibration curve on Roche Integra 400.

FIG. 5 illustrates an exemplary 25(OH)D homogeneous enzyme immunoassay calibration curve on Mindray BS-800.

FIG. 6 illustrates an exemplary 25(OH)D homogeneous enzyme immunoassay calibration curve on Horiba Pentra 400.

FIG. 7 illustrates exemplary linearity result obtained from Horiba Pentra 400 (two cuvettes format).

FIG. 8 illustrates exemplary linearity result obtained from Roche Integra 400 (one cuvette format).

FIG. 9 illustrates an exemplary calibration curve obtained from Roche Modular P (one cuvette format).

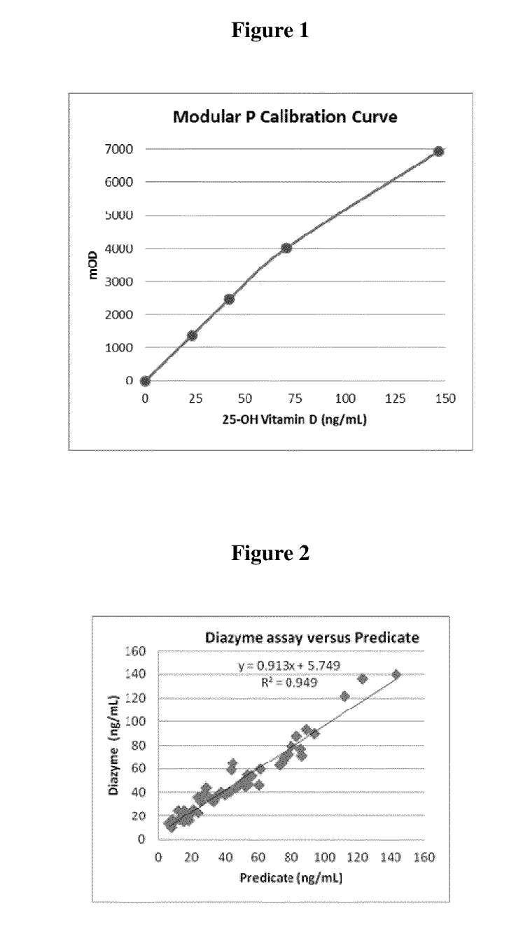

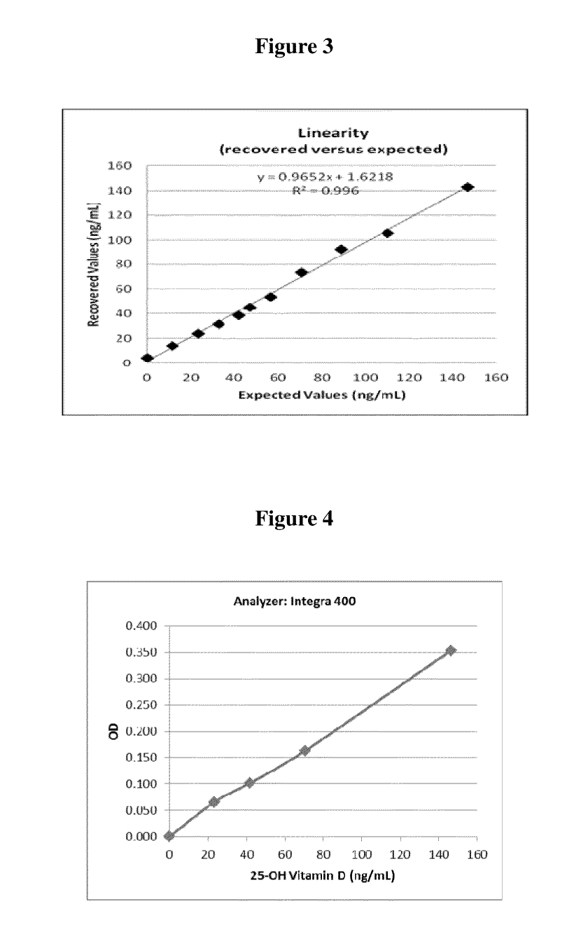

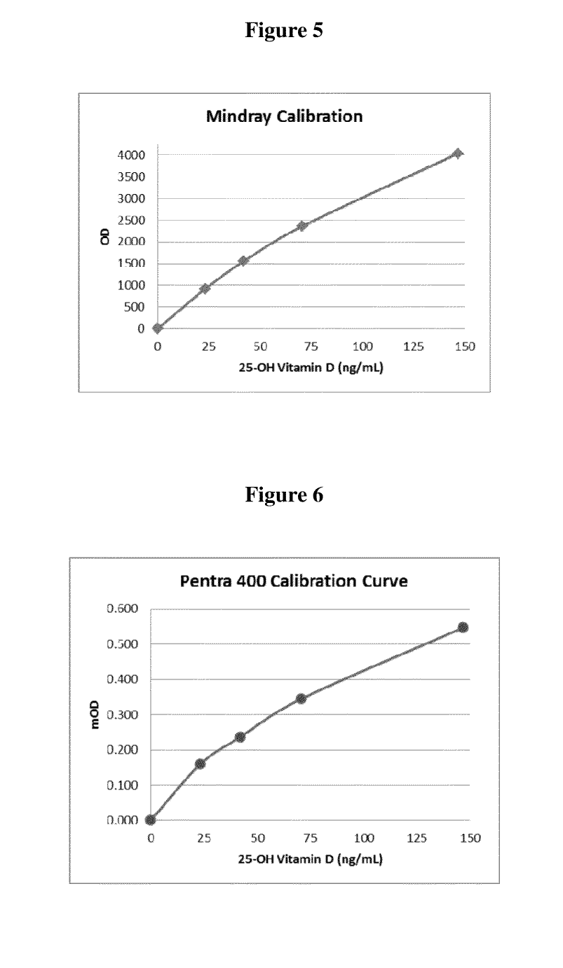

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “vitamin D moiety” refers to all members or forms of the Vitamin D family which is a group of fat-soluble secosteroids responsible for intestinal absorption of calcium and phosphate. Exemplary vitamin D forms include vitamin D₁, D₂ (ergocalciferol), D₃ (cholecalciferol), D₄, and D₅. Exemplary vitamin D moieties also include calcidiol, which is also known as calcifediol (INN), 25-hydroxycholecalciferol, or 25-hydroxyvitamin D-abbreviated 25(OH)D; and which is the specific vitamin D metabolite that is measured in serum to determine a person's vitamin D status, and calcitriol, the biologically active form of vitamin D.

As used herein, a “binding partner (or binder)” refers to any substance that binds to a target or an analyte, e.g., a vitamin D moiety, with desired affinity and/or specificity. Non-limiting examples of the binding reagent include cells, cellular organelles, viruses, particles, microparticles, molecules, e.g., antibody, or an aggregate or complex thereof, or an aggregate or complex of molecules.

As used herein, “antibody” includes not only intact polyclonal or monoclonal antibodies, but also fragments thereof (such as Fab, Fab′, F(ab′)₂, Fv), single chain (ScFv), a diabody, a multi-specific antibody formed from antibody fragments, mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class.

As used herein, the term “specifically binds” refers to the specificity of a binding reagent, e.g., an antibody, such that it preferentially binds to a defined analyte or target e.g., a vitamin D moiety. Recognition by a binding reagent or an antibody of a particular analyte or target in the presence of other potential targets is one characteristic of such binding. In some embodiments, a binding reagent that specifically binds to an analyte avoids binding to other interfering moiety or moieties in the sample to be tested.

As used herein the term “avoids binding” refers to the specificity of particular binding reagents, e.g., antibodies or antibody fragments. Binding reagents, antibodies or antibody fragments that avoid binding to a particular moiety generally contain a specificity such that a large percentage of the particular moiety would not be bound by such binding reagents, antibodies or antibody fragments. This percentage generally lies within the acceptable cross reactivity percentage with interfering moieties of assays utilizing the binding reagents or antibodies directed to detecting a specific target. Frequently, the binding reagents, antibodies or antibody fragments of the present disclosure avoid binding greater than about 90% of an interfering moiety, although higher percentages are clearly contemplated and preferred. For example, binding reagents, antibodies or antibody fragments of the present disclosure avoid binding about 91%, about 92%, about 93%, about 94%, about 95%, about 96%, about 97%, about 98%, about 99%, and about 99% or more of an interfering moiety. Less occasionally, binding reagents, antibodies or antibody fragments of the present disclosure avoid binding greater than about 50%, greater than about 60%, greater than about 70%, or greater than about 75%, or greater than about 80%, or greater than about 85% of an interfering moiety.

A “vitamin D binding protein” is also known as gc-globulin (group-specific component). As used herein, a “vitamin D binding protein” refers to a Vitamin D-binding protein in the albumin family. A “vitamin D binding protein” is often found in plasma, ascitic fluid, cerebrospinal fluid and/or on the surface of many cell types. A “vitamin D binding protein” often binds to vitamin D and its metabolites and transports them to target tissues in vivo. An exemplary “vitamin D binding protein” in humans is encoded by the GC gene.

As used herein the term “assessing” is intended to include quantitative and qualitative determination in the sense of obtaining an absolute value for the amount or concentration of the analyte present in the sample, and also of obtaining an index, ratio, percentage, visual or other value indicative of the level of analyte in the sample. Assessment may be direct or indirect and the chemical species actually detected need not of course be the analyte itself but may for example be a derivative thereof or some further substance.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “blood sample” refers to a whole blood sample or a plasma or serum fraction derived therefrom. Preferably, the blood sample refers to a human blood sample such as whole blood or a plasma or serum fraction derived therefrom. Also preferably, the blood sample is pre-treated before the assay by removing substantially all hemoglobin (i.e., red blood cells) in order to eliminate or significantly reduce the oxidative interference from the hemoglobin molecules.

As used herein the term “whole blood” refers to a blood sample that has not been fractionated and contains both cellular and fluid components. As used herein, “whole blood” refers to freshly drawn blood which is tested before it clots, or a conventionally-drawn blood sample, which may be drawn into a vacutainer, and which may contain an anticoagulant, such as lithium-heparin, EDTA, etc., or to which one or more other standard clinical agents may be added in the course of routine clinical testing.

As used herein, the term “plasma” refers to the fluid, non-cellular component of the whole blood. Depending on the separation method used, plasma may be completely free of cellular components, or may contain various amounts of platelets and/or a small amount of other cellular components. Because plasma includes various clotting factors such as fibrinogen, the term “plasma” is distinguished from “serum” as set forth below.

As used herein, the term “serum” refers to whole mammalian serum, such as whole human serum. Further, as used herein, “serum” refers to blood plasma from which clotting factors (e.g., fibrinogen) have been removed.

As used herein, the term “fluid” refers to any composition that can flow. Fluids thus encompass compositions that are in the form of semi-solids, pastes, solutions, aqueous mixtures, gels, lotions, creams and other such compositions.

As used herein, the term “disease” or “disorder” refers to a pathological condition in an organism resulting from, e.g., infection or genetic defect, and characterized by identifiable symptoms.

As used herein, “contacting” means bringing two or more components together. “Contacting” can be achieved by mixing all the components in a fluid or semi-fluid mixture. “Contacting” can also be achieved when one or more components are brought into contact with one or more other components on a solid surface such as a solid tissue section or a substrate.

As used herein, the term “comparing” generally means examining in order to note similarities or differences between two or more values. Preferably, “comparing” refers to quantitative comparisons such as, for example, subtracting one value from another, calculating a ratio of two values, calculating a percentage of one value with respect to another, or combining these types of calculations to produce a single number. As used herein, “comparing” further refers to comparisons made by a human, comparisons made by a computer or other processor, and comparisons made by a human in combination with a computer or other processor.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range

Other objects, advantages and features of the present invention will become apparent from the following specification taken in conjunction with the accompanying drawings.

### B. METHODS FOR ASSAYING A VITAMIN D MOIETY

In one aspect, the present invention provides methods for a method for assaying a vitamin D moiety in a sample, which method comprises: a) contacting a sample containing or suspected of containing a vitamin D moiety with 1) a buffer of acidic pH, 2) a specific binding partner that specifically binds to said vitamin D moiety, if present in said sample, to form a vitamin D moiety/specific binding partner complex, said binding partner being different from a natural vitamin D binding protein for said vitamin D moiety, 3) an enzyme donor (ED)-vitamin D moiety conjugate, said enzyme donor (ED) comprising a first fragment of a β-galactosidase, and 4) an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase, said sample is contacted with said buffer of acidic pH and said specific binding partner in one or more steps, and said sample is contacted with said ED-vitamin D moiety conjugate and said enzyme acceptor (EA) in other separate one or more steps; and b) assessing binding between said specific binding partner and said vitamin D moiety to determine the presence, absence and/or amount of said vitamin D moiety in said sample by measuring the activity of said reassembled active β-galactosidase in the presence of a β-galactosidase substrate. Preferably, the above method utilizes the cloned enzyme donor immunoassay (CEDIA) principle.

In some embodiments, the present methods do not comprise a step of removing protein from the sample (See e.g., U.S. Pat. No. 5,821,020), such as the natural vitamin D binding protein for the vitamin D moiety, prior to assessing binding between the specific binding partner and the vitamin D moiety. In other embodiments, the present methods do not comprise any wash step.

The present methods can be conducted in any suitable assay format. In some embodiments, the present methods are conducted as a homogeneous assay. In other embodiments, the present methods are conducted as a heterogeneous assay.

The present methods can be used for assaying any suitable vitamin D moiety in a sample. In some embodiments, the vitamin D moiety is vitamin D₃, vitamin D₂, a vitamin D metabolite, 1,25-dihydroxyvitamin D₃ [1,25(OH)₂D₃], or 3-epi-25-hydroxyvitamin D3 (3-epi-25(OH)D3 or C3-epimer). In other embodiments, the vitamin D moiety is 25-hydroxy-vitamin D (25(OH)D), e.g., 25(OH)D3, 25(OH)D2 or a sum of 25(OH)D2 and 25(OH)D3. In still other embodiments, the 25(OH)D is a sum of 25(OH)D2 and 25(OH)D3.

The buffer of acidic pH can have any suitable acidic pH range. In some embodiments, the buffer of acidic pH has a pH ranging from about 1.0 to about 5.0, e.g., 1.0, 1.5, 2.0, 2.5, 3.0, 3.5 and 4.0. In other embodiments, the buffer of acidic pH has a pH ranging from about 2.0 to about 4.0, e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0.

Any suitable specific binding partner that specifically binds to the vitamin D moiety can be used in the present methods. In some embodiments, the specific binding partner that specifically binds to the vitamin D moiety is an antibody that specifically binds to the vitamin D moiety. In other embodiments, the antibody specifically binds to 25(OH)D, e.g., antibody specifically binds to 25(OH)D2 and/or 25(OH)D3, or an antibody specifically binds to 25(OH)D 2 and 25(OH)D 3 with the same or a similar binding affinity. Antibodies in any suitable forms can be used. For example, a polyclonal antibody or a monoclonal antibody can be used. In still other embodiments, exemplary antibodies disclosed in U.S. patent publication No. 2011/0097733 A1 can be used.

In some embodiments ‘ED” refers to the enzyme donor commonly used in the CEDIA assay technique (Cloned Enzyme Donor Immunoassay) as described in the literature (e.g., Clin. Chem. 32, 1986 by D. R. Henderson, et al.) and described in patents, e.g., U.S. Pat. No. 4,708,929. Similarly, in some embodiments “EA” refers to the enzyme acceptor commonly used in the CEDIA assay technology as described in the art, e.g., as described by D. R. Henderson in U.S. Pat. No. 4,708,929, and in the published article (e.g., Clin. Chem. 32, 1986). The assay principle of the CEDIA technique is based on α-complementation of the enzyme β-galactosidase and the binding competition between the enzyme donor-analyte conjugates and analytes in the sample towards the specific analyte binding partner (e.g., antibody).

In some embodiments, the β-galactosidase used can be of microorganism origin, such as E. coli. The E. coli β-galactosidase can be spilt into two inactive fragments using genetic engineering methodology (see, e.g., Henderson, et al., 1986). The larger fragment containing approximately 990 amino acid residues is termed enzyme acceptor (EA) which contains a deletion near the amino terminus of approximately 5%-10% of the β-galactosidase single subunit. The smaller fragment containing approximately 40-100 amino acid residues is termed enzyme donor (ED) which contains the sequence missing from EA. When mixed together, these fragments spontaneously re-associate to form intact enzyme subunits which then assemble to form tetramers and express β-galactosidase activity. This process is termed α-complementation (see, e.g., Zabin I, beta-galactosidase alpha-complementation, A method of protein-protein interaction. Mol. Cell Biochem., 49:87-96 (1982)).

In some embodiments, for a precise conjugation position on the ED molecule, the sequence of ED can be modified to contain a cysteine or lysine residue as a specific site for covalent attachment of a hapten or an analyte analogue, which does not affect the complementation of EA with ED to form active enzyme. The conjugation site can also be chosen so that binding of antibody to the hapten blocks complementation of ED with EA. Analyte present in a sample will compete for binding to the limited number of antibody sites, making ED-analyte conjugate available for formation of active enzyme. Thus, the amount of enzyme formed, which can be measured using any suitable methods, e.g., spectrophotometrically, by the kinetic rate of chromogenic substrate hydrolysis, is directly proportional to the analyte concentration in the sample.

Any suitable vitamin D moiety can be used in the ED-vitamin D moiety conjugate. In some embodiments, the vitamin D moiety to be assayed and the vitamin D moiety in the ED-vitamin D moiety conjugate can have the same or a similar affinity towards the vitamin D binding partner used in the method. For example, the vitamin D moiety in the ED-vitamin D moiety conjugate can be vitamin D₃, vitamin D₂, a vitamin D metabolite or 1,25-dihydroxyvitamin D₃ (1,25-(OH)₂D₃). The vitamin D metabolite can be 25-hydroxy-vitamin D (25(OH)D), e.g., 25(OH)D3, 25(OH)D2, or a combination thereof. In some embodiments, the vitamin D moiety to be assayed and the vitamin D moiety in the ED-vitamin D moiety conjugate are the same.

The first fragment of a β-galactosidase in the enzyme donor (ED) and/or the second fragment of a β-galactosidase in the enzyme acceptor (EA) can be prepared in any suitable manner or methods. In some embodiments, the first fragment of a β-galactosidase in the enzyme donor (ED) and the second fragment of a β-galactosidase in the enzyme acceptor (EA) are prepared separately. In other embodiments, the first fragment of a β-galactosidase in the enzyme donor (ED) and the second fragment of a β-galactosidase in the enzyme acceptor (EA) are prepared simultaneously.

The first fragment of a β-galactosidase in the enzyme donor (ED) and/or the second fragment of a β-galactosidase in the enzyme acceptor (EA) can be derived and/or obtained from any suitable β-galactosidase. For example, the first fragment of a β-galactosidase in the enzyme donor (ED) and/or the second fragment of a β-galactosidase in the enzyme acceptor (EA) can be derived and/or obtained from E. coli wild type β-galactosidase or its mutants.

In some embodiments, the second fragment of a β-galactosidase in the enzyme acceptor (EA) can comprise a deletion near the amino terminus of about 5%-10%, e.g., 5%, 6%, 7%, 8%, 9%, or 10%, of the β-galactosidase single subunit. In other embodiments, the second fragment of a β-galactosidase in the enzyme acceptor (EA) can comprise about 990 to about 1,010 amino acid residues, e.g., the second fragment of a β-galactosidase in the enzyme acceptor (EA) being comprised within amino acid residues 20 and 1024 of E. coli β-galactosidase.

In some embodiments, the first fragment of a β-galactosidase in the enzyme donor (ED) can comprise about 40 to 100 amino acid residues, e.g., about 40, 50, 60, 70, 80, 90 or 100 amino acid residues, of the β-galactosidase that are missing from the second fragment of a β-galactosidase in the enzyme acceptor (EA), e.g., the first fragment of a β-galactosidase in the enzyme donor (ED) being comprised within amino acid residues 1 and 100 of E. coli β-galactosidase.

The first fragment of a β-galactosidase in the enzyme donor (ED) can comprise any suitable modification(s). In some embodiments, the first fragment of a β-galactosidase in the enzyme donor (ED) can be modified to contain a cysteine residue for covalent attachment to the vitamin D moiety.

A sample can be contacted with the various agents, including the buffer of acidic pH, the specific binding partner, the ED-vitamin D moiety conjugate and/or the enzyme acceptor (EA), in any suitable manner or order. For example, a sample can be contacted with a buffer of acidic pH and a vitamin D specific binding partner in the same step, or in different steps. In another example, a sample can be contacted with an ED-vitamin D moiety conjugate and an enzyme acceptor (EA) in the same step, or in different steps. A sample, however, should be contacted with a buffer of acidic pH and/or a vitamin D specific binding partner, and an ED-vitamin D moiety conjugate and/or an enzyme acceptor (EA), in different steps.

In some embodiments, a sample is contacted with the buffer of acidic pH and the specific binding partner before the sample is contacted with the ED-vitamin D moiety conjugate and/or the enzyme acceptor (EA). For example, the sample can be first contacted by the vitamin D binding partner (e.g., antibody or antibodies) and the acidic pH buffer solution prior to be contacted by the ED-vitamin D moiety conjugate (e.g., ED-25(OH)D conjugate) and the enzyme acceptor (EA) simultaneously. In another example, the sample can be first contacted by the vitamin D binding partner (e.g., antibody or antibodies) and the acidic pH buffer solution prior to be contacted by the ED-vitamin D moiety conjugate (e.g., ED-25(OH)D conjugate) and the enzyme acceptor (EA) sequentially. In still another example, the sample can be contacted with the vitamin D binding partner (e.g., antibody or antibodies) and the acidic pH buffer solution, then contacted with the ED-vitamin D moiety conjugate (e.g., ED-25(OH)D conjugate), and then contacted with the enzyme acceptor (EA).

In some embodiments, the additions of the sample and reagents can follow a specific sequence or order in which: 1) the sample is first diluted with a buffer comprising a vitamin D binding partner (e.g., antibody or antibodies), and part of the diluted sample is then contacted with the acidic buffer solution comprising a β-galactosidase substrate prior to the additions of the ED-vitamin D moiety conjugate (e.g., ED-25(OH)D conjugate) and the enzyme acceptor (EA); and 2) the ED-vitamin D moiety conjugate (e.g., ED-25(OH)D conjugate) and the enzyme acceptor (EA) are added into the reaction mixture sequentially or in two separated steps.

In other embodiments, the additions of the sample and reagents can follow a specific sequence or order in which: 1) the sample is first contacted by the acidic buffer solution comprising a specific Vitamin D binding partner (e.g., antibody or antibodies) and a β-galactosidase substrate prior to the additions of the ED-vitamin D moiety conjugate (e.g., ED-25(OH)D conjugate) and the enzyme acceptor (EA); and 2) the ED-vitamin D moiety conjugate (e.g., ED-25(OH)D conjugate) and the enzyme acceptor (EA) are added into the reaction mixture sequentially or in two separated steps.

In some embodiments, the present method can be a homogenous assay that is conducted in a single reaction container (e.g., a cuvette) comprising the steps of sample dilution with a buffer comprising the vitamin D binding partner, the acidic pH buffer solution comprising a β-galactosidase substrate, the ED-vitamin D moiety conjugate (e.g., ED-25(OH)D conjugate), the enzyme acceptor (EA) and one or more stabilizers. Any suitable stabilizer(s) can be used. For example, the one or more stabilizers can be a simple polyol (sugar alcohol) compound, e.g., glycerol, a sugar alcohol, e.g., sorbitol and/or a reducing agent, e.g., TCEP.

In some embodiments, the present method can be a homogenous assay that is conducted in two separated containers (e.g., two cuvettes) with the sample being first diluted with a buffer comprising the vitamin D binding partner in one cuvette (sample dilution cuvette) followed by mixing part of the diluted sample with the acidic pH buffer solution comprising a β-galactosidase substrate in another separated cuvette (reaction cuvette) before additions of the ED-vitamin D moiety conjugate (e.g., ED-25(OH)D conjugate), and the enzyme acceptor (EA).

Any suitable β-galactosidase substrate can be used in the present methods. For example, the β-galactosidase substrate can be o-nitrophenyl-β-D-galactoside (ONPG), chlorophenol red-β-D-galactopyranoside (CPRG), or an analogue thereof. A suitable β-galactosidase substrate can be hydrolyzed by a β-galactosidase into a galactopyranoside and a chromophore. Often, the chromophore can be monitored for determination of the β-galactosidase activity.

The present methods can be conducted in any suitable time frame. In some embodiments, the present methods have a total assay time that is at about 30 minutes or shorter, e.g., about 29, 28, 27, 26, 25, 24, 23, 22, 21, 20, 19, 18, 17, 16, 15, 14, 13, 12, 11, 10, 9, 8, 7, 6, 5, 4, 3 minutes.

The pH in a final reaction mixture comprising the sample, the acidic pH buffer, the vitamin D binding partner (e.g., antibody or antibodies), the ED-25(OH)D conjugate, the EA protein and the β-galactosidase substrate can be at any suitable value or range. In some embodiments, the pH in a final reaction mixture comprising the sample, the acidic pH buffer, the vitamin D binding partner (e.g., antibody or antibodies), the ED-25(OH)D conjugate, the EA protein and the β-galactosidase substrate is at 5 or higher. In other embodiments, the pH in a final reaction mixture containing entire reaction components is at 10 or lower. In still other embodiments, the pH in a final reaction mixture comprising the sample, the acidic pH buffer solution, the vitamin D binding partner (e.g., antibody or antibodies), the ED-25(OH)D conjugate, the EA protein, the β-galactosidase substrate and stabilizers is in a range from about 4 to about 13, e.g., at about 4, 4.5, 5, 5.5, 5.6, 5.7, 5.8, 5.9, 6, 6.5, 7, 7.5, 8, 8.5, 9, 9.5, 10, 10.5, 11, 11.5, 12, 12.5, or 13.

The present methods can be conducted on any suitable analytic instruments. In some embodiments, the present methods are conducted on a general chemistry analyzer or a clinical chemistry analyzer, e.g., general chemistry analyzer or clinical chemistry analyzer from Roche, Modular P, Cobas series, Hitachi series, Mindray BS series, Horiba Pentra, alpha Wassermann ACE system, and Siemens Dimension. In other embodiments, the general chemistry analyzer or the clinical chemistry analyzer can be capable of taking at least 3 reagents in an assay.

The present methods can be used for any suitable purpose. In some embodiments, the present methods can be used to assess status of the vitamin D moiety in a subject, and the sample is a biological sample obtained and/or derived from the subject. The present methods can be used for assess status of the vitamin D moiety in any suitable subject, e.g., a mammal, a non-human mammal, a human or an experimental animal.

The present methods can be used for assaying a vitamin D moiety in any suitable sample. In some embodiments, the sample is a biological fluid, e.g., whole blood, plasma, serum or urine.

### C. KITS FOR ASSAYING A VITAMIN D MOIETY AND USES THEREOF

In another aspect, the present invention provides a kit for assaying a vitamin D moiety in a sample, which kit comprises: a) a buffer of acidic pH, b) a specific binding partner that specifically binds to a vitamin D moiety, if present in said sample, to form a vitamin D moiety/specific binding partner complex, said binding partner being different from a natural vitamin D binding protein for said vitamin D moiety, c) an enzyme donor (ED)-vitamin D moiety conjugate, said enzyme donor (ED) comprising a first fragment of a β-galactosidase, and d) an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

Any suitable specific binding partner that specifically binds to the vitamin D moiety can be used in the present kits. In some embodiments, the specific binding partner that specifically binds to the vitamin D moiety is an antibody that specifically binds to the vitamin D moiety. In other embodiments, the antibody specifically binds to 25(OH)D. Antibodies in any suitable forms can be used. For example, a polyclonal antibody or a monoclonal antibody can be used. In still other embodiments, exemplary antibodies disclosed in U.S. patent publication No. 2011/0097733 A1 can be used.

The present kits can be used for assaying any suitable vitamin D moiety in a sample. In some embodiments, the vitamin D moiety is vitamin D₃, vitamin D₂, a vitamin D metabolite, 1,25-dihydroxyvitamin D₃ [1,25(OH)₂D₃], or 3-epi-25-hydroxyvitamin D3 (3-epi-25(OH)D3 or C3-epimer). In other embodiments, the vitamin D moiety is 25-hydroxy-vitamin D (25(OH)D), e.g., 25(OH)D3, 25(OH)D2 or a sum of 25(OH)D2 and 25(OH)D3. In still other embodiments, the 25(OH)D is a sum of 25(OH)D2 and 25(OH)D3.

The buffer of acidic pH can have any suitable acidic pH range. In some embodiments, the buffer of acidic pH has a pH ranging from about 1.0 to about 5.0, e.g., 1.0, 1.5, 2.0, 2.5, 3.0, 3.5 and 4.0. In other embodiments, the buffer of acidic pH has a pH ranging from about 2.0 to about 4.0, e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0.

Any suitable vitamin D moiety can be used in the ED-vitamin D moiety conjugate. In some embodiments, the vitamin D moiety to be assayed and the vitamin D moiety in the ED-vitamin D moiety conjugate can have the same or a similar affinity towards the vitamin D binding partner used in the method. For example, the vitamin D moiety in the ED-vitamin D moiety conjugate can be vitamin D₃, vitamin D₂, a vitamin D metabolite or 1,25-dihydroxyvitamin D₃ (1,25-(OH)₂D₃). The vitamin D metabolite can be 25-hydroxy-vitamin D (25(OH)D), e.g., 25(OH)D3, 25(OH)D2, or a combination thereof. In some embodiments, the vitamin D moiety to be assayed and the vitamin D moiety in the ED-vitamin D moiety conjugate are the same.

The first fragment of a β-galactosidase in the enzyme donor (ED) and/or the second fragment of a β-galactosidase in the enzyme acceptor (EA) can be prepared in any suitable manner or methods. In some embodiments, the first fragment of a β-galactosidase in the enzyme donor (ED) and the second fragment of a β-galactosidase in the enzyme acceptor (EA) are prepared separately. In other embodiments, the first fragment of a β-galactosidase in the enzyme donor (ED) and the second fragment of a β-galactosidase in the enzyme acceptor (EA) are prepared simultaneously.

The first fragment of a β-galactosidase in the enzyme donor (ED) and/or the second fragment of a β-galactosidase in the enzyme acceptor (EA) can be derived and/or obtained from any suitable β-galactosidase. For example, the first fragment of a β-galactosidase in the enzyme donor (ED) and/or the second fragment of a β-galactosidase in the enzyme acceptor (EA) can be derived and/or obtained from E. coli wild type β-galactosidase or its mutants.

In some embodiments, the second fragment of a β-galactosidase in the enzyme acceptor (EA) can comprise a deletion near the amino terminus of about 5%-10%, e.g., 5%, 6%, 7%, 8%, 9%, or 10%, of the β-galactosidase single subunit. In other embodiments, the second fragment of a β-galactosidase in the enzyme acceptor (EA) can comprise about 990 to about 1,010 amino acid residues, e.g., the second fragment of a β-galactosidase in the enzyme acceptor (EA) being comprised within amino acid residues 20 and 1024 of E. coli β-galactosidase.

In some embodiments, the first fragment of a β-galactosidase in the enzyme donor (ED) can comprise about 40 to 100 amino acid residues, e.g., about 40, 50, 60, 70, 80, 90 or 100 amino acid residues, of the β-galactosidase that are missing from the second fragment of a β-galactosidase in the enzyme acceptor (EA), e.g., the first fragment of a β-galactosidase in the enzyme donor (ED) being comprised within amino acid residues 1 and 100 of E. coli β-galactosidase.

The first fragment of a β-galactosidase in the enzyme donor (ED) can comprise any suitable modification(s). In some embodiments, the first fragment of a β-galactosidase in the enzyme donor (ED) can be modified to contain a cysteine residue for covalent attachment to the vitamin D moiety.

The present kits can comprise any additional suitable reagents or components. In some embodiments, the present kits further comprise means for assessing binding between the specific binding partner and the vitamin D moiety to determine the presence, absence and/or amount of the vitamin D moiety in the sample. For example, the means for assessing binding between the specific binding partner and the vitamin D moiety can comprise a β-galactosidase substrate or a vitamin D calibrator. Any suitable β-galactosidase substrate can be used in the present kits. For example, the β-galactosidase substrate can be o-nitrophenyl-β-D-galactoside (ONPG), chlorophenol red-β-D-galactopyranoside (CPRG), or an analogue thereof.

In some embodiments, the β-galactosidase substrate can be comprised in the buffer of acidic pH. In other embodiments, the present kits can comprise a set of vitamin D calibrators of known vitamin D values.

The reagents or components in the present kits can be formulated or arranged in any suitable fashion or form. In some embodiments, the present kits can comprise the following reagents: a) a sample dilution buffer comprising the specific vitamin D binding partner (e.g., antibody or antibodies); b) a first assay reagent (R1) comprising a β-galactosidase substrate in the buffer of acidic pH; c) a second assay reagent (R2) comprising the ED-vitamin D moiety conjugate (e.g., ED-25(OH)D conjugate); and d) a third assay reagent (R3) comprising the enzyme acceptor (EA). The sample dilution buffer, R1, R2 and R3 can be stored in any suitable format. For example, the present kits can comprise the sample dilution buffer, R1, R2 and R3 in a liquid stable format. In another example, the present kits can further comprise a vitamin D calibrator, or a set of vitamin D calibrators of known vitamin D values.

The above kits can be used in a method for assaying a vitamin D moiety in a sample, which method comprises: a) diluting a sample with the sample dilution buffer comprising the specific vitamin D binding partner, mixing part of the diluted sample with R1, incubating the mixture for a period of time, adding R2, and after another period of incubation time, adding R3 to the reaction mixture; and b) quantifying the amount of a vitamin D moiety (e.g., 25(OH)D) in the sample by measuring the optical change of the reaction mixture and using a set of vitamin D calibrators of known vitamin D values (e.g., a set of 25(OH)D calibrators).

In other embodiments, the present kits can comprise the following reagents: a) a first assay reagent (R1) comprising an acidic pH buffer solution comprising a β-galactosidase substrate and a specific binding partner that specifically binds to said vitamin D moiety, if present in said sample, said binding partner being different from a natural vitamin D binding protein for said vitamin D moiety; b) a second assay reagent (R2) comprising an enzyme donor (ED)-vitamin D moiety conjugate, said enzyme donor (ED) comprising a first fragment of a β-galactosidase; and c) a third assay reagent (R3) comprising an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase. The R1, R2 and R3 reagents can be stored in any suitable format. For example, the present kits can comprise R1, R2 and R3 in a liquid stable format or in a solid format, e.g., lyophilized format, or in a mixed format of a liquid and solid, e.g., lyophilized. In another example, the present kits can further comprise a vitamin D calibrator, or a set of vitamin D calibrators of known vitamin D values.

The above kits can be used in a method for assaying a vitamin D moiety in a sample, which method comprises: a) mixing a sample with R1 to form a reaction mixture; b) after a period of incubation, adding R2 and R3 to the reaction mixture in two separate steps; c) quantifying the amount of a vitamin D moiety (e.g., 25(OH)D) in the sample by measuring the optical change of the reaction mixture and using a set of vitamin D calibrators of known vitamin D values (e.g., a set of 25(OH)D calibrators).

### D. REACTION MIXTURES FOR ASSAYING A VITAMIN D MOIETY IN A SAMPLE

In still another aspect, the present invention provides a reaction mixture for assaying a vitamin D moiety in a sample, which reaction mixture comprises: a) a specific binding partner that specifically binds to a vitamin D moiety, if present in said sample, to form a vitamin D moiety/specific binding partner complex, said binding partner being different from a natural vitamin D binding protein for said vitamin D moiety, b) an enzyme donor (ED)-vitamin D moiety conjugate, said enzyme donor (ED) comprising a first fragment of a β-galactosidase, and c) an enzyme acceptor (EA), said enzyme acceptor (EA) comprising a second fragment of a β-galactosidase, wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase. Preferably, the present reaction mixture exists in an acidic environment.

Any suitable specific binding partner that specifically binds to the vitamin D moiety can be used in the present reaction mixtures. In some embodiments, the specific binding partner that specifically binds to the vitamin D moiety is an antibody that specifically binds to the vitamin D moiety. In other embodiments, the antibody specifically binds to 25(OH)D. Antibodies in any suitable forms can be used. For example, a polyclonal antibody or a monoclonal antibody can be used. In still other embodiments, exemplary antibodies disclosed in U.S. patent publication No. 2011/0097733 A1 can be used.

Any suitable vitamin D moiety can be used in the ED-vitamin D moiety conjugate. In some embodiments, the vitamin D moiety to be assayed and the vitamin D moiety in the ED-vitamin D moiety conjugate can have the same or a similar affinity towards the vitamin D binding partner used in the method. For example, the vitamin D moiety in the ED-vitamin D moiety conjugate can be vitamin D₃, vitamin D₂, a vitamin D metabolite, 1,25-dihydroxyvitamin D₃ (1,25-(OH)₂D₃), or 3-epi-25-hydroxyvitamin D3 (3-epi-25(OH)D3 or C3-epimer). The vitamin D metabolite can be 25-hydroxy-vitamin D (25(OH)D), e.g., 25(OH)D3, 25(OH)D2, or a combination thereof. In some embodiments, the vitamin D moiety to be assayed and the vitamin D moiety in the ED-vitamin D moiety conjugate are the same.

The first fragment of a β-galactosidase in the enzyme donor (ED) and/or the second fragment of a β-galactosidase in the enzyme acceptor (EA) can be prepared in any suitable manner or methods. In some embodiments, the first fragment of a β-galactosidase in the enzyme donor (ED) and the second fragment of a β-galactosidase in the enzyme acceptor (EA) are prepared separately. In other embodiments, the first fragment of a β-galactosidase in the enzyme donor (ED) and the second fragment of a β-galactosidase in the enzyme acceptor (EA) are prepared simultaneously.

The first fragment of a β-galactosidase in the enzyme donor (ED) and/or the second fragment of a β-galactosidase in the enzyme acceptor (EA) can be derived and/or obtained from any suitable β-galactosidase. For example, the first fragment of a β-galactosidase in the enzyme donor (ED) and/or the second fragment of a β-galactosidase in the enzyme acceptor (EA) can be derived and/or obtained from E. coli wild type β-galactosidase or its mutants.

In some embodiments, the second fragment of a β-galactosidase in the enzyme acceptor (EA) can comprise a deletion near the amino terminus of about 5%-10%, e.g., 5%, 6%, 7%, 8%, 9%, or 10%, of the β-galactosidase single subunit. In other embodiments, the second fragment of a β-galactosidase in the enzyme acceptor (EA) can comprise about 990 to about 1,010 amino acid residues, e.g., the second fragment of a β-galactosidase in the enzyme acceptor (EA) being comprised within amino acid residues 20 and 1024 of E. coli β-galactosidase.

In some embodiments, the first fragment of a β-galactosidase in the enzyme donor (ED) can comprise about 40 to 100 amino acid residues, e.g., about 40, 50, 60, 70, 80, 90 or 100 amino acid residues, of the β-galactosidase that are missing from the second fragment of a β-galactosidase in the enzyme acceptor (EA), e.g., the first fragment of a β-galactosidase in the enzyme donor (ED) being comprised within amino acid residues 1 and 100 of E. coli β-galactosidase.

The first fragment of a β-galactosidase in the enzyme donor (ED) can comprise any suitable modification(s). In some embodiments, the first fragment of a β-galactosidase in the enzyme donor (ED) can be modified to contain a cysteine residue for covalent attachment to the vitamin D moiety.

The present reaction mixtures can comprise any additional suitable reagents or components. In some embodiments, the reaction mixture further comprises a vitamin D moiety that is bound to the specific binding partner.

The present reaction mixtures can be formulated or arranged in any suitable fashion or manner. In some embodiments, the present reaction mixtures are contained in a single phase. In other embodiments, the present reaction mixtures are contained in multiple phases, e.g., two or three phases.

The pH in a final reaction mixture comprising the sample, the acidic pH buffer, the vitamin D binding partner (antibody or antibodies), the ED-25(OH)D conjugate, the EA protein and the β-galactosidase substrate can be at any suitable value or range. In some embodiments, the pH in a final reaction mixture comprising the sample, the acidic pH buffer, the vitamin D binding partner (antibody or antibodies), the ED-25(OH)D conjugate, the EA protein and the β-galactosidase substrate is at 5 or higher. In other embodiments, the pH in a final reaction mixture containing entire reaction components is at 10 or lower. In still other embodiments, the pH in a final reaction mixture comprising the sample, the acidic pH buffer solution, the vitamin D binding partner (antibody or antibodies), the ED-25(OH)D conjugate, the EA protein, the β-galactosidase substrate and stabilizers is in a range from about 4 to about 13, e.g., at about 4, 4.5, 5, 5.5, 5.6, 5.7, 5.8, 5.9, 6, 6.5, 7, 7.5, 8, 8.5, 9, 9.5, 10, 10.5, 11, 11.5, 12, 12.5, or 13.

### E. EXEMPLARY EMBODIMENTS

In some embodiments, the present disclosure provides for a homogeneous method for assaying a vitamin D moiety in a sample, which method utilizes the cloned enzyme donor immunoassay (CEDIA) principle (see e.g., D. R. Henderson, et al., Clin. Chem. 32, 1986) and comprises the steps of: a) contacting a sample containing or suspected of containing a vitamin D moiety with a buffer of acidic pH and a vitamin D specific antibody or antibodies; b) after a period of incubation of the step (a), an enzyme donor (ED)-vitamin D (ED-25(OH)D) conjugate is added to the reaction mixture of the step (a); and c) assessing binding between said vitamin D antibody and said vitamin D moiety to determine the presence, absence and/or amount of said vitamin D moiety in said sample by addition of the enzyme acceptor (EA) that forms an active β-galactosidase with the unbound ED-25(OH)D conjugate through the α-complementation. The active β-galactosidase catalyzes the hydrolysis of the enzyme substrate that is present in the reaction mixture. The β-galactosidase enzyme activity detected from the reaction mixture is proportional to the amount of vitamin D in the sample, and the amount of vitamin D can be quantified by using a calibration curve constructed with a set of known vitamin D value samples (calibrators).

In other embodiments, the present disclosure provides for kits for assaying a vitamin D moiety in a sample, which kits comprise: a) a buffer of acidic pH; b) a specific binding partner that specifically binds to said vitamin D moiety, if present in said sample, said binding partner being different from a natural vitamin D binding protein for said vitamin D moiety; c) a ED-25(OH)D conjugate; and d) an EA protein, and optionally a β-galactosidase substrate.

In still other embodiments, the present disclosure provides for reaction mixtures for assaying a vitamin D moiety in a sample, which reaction mixtures comprise: a) a buffer of acidic pH; b) a specific binding partner that specifically binds to said vitamin D moiety, if present in said sample, said binding partner being different from a natural vitamin D binding protein for said vitamin D moiety; and c) a ED-25(OH)D conjugate, and d) a EA protein, and optionally a β-galactosidase substrate such as a ONPG (o-nitrophenyl-β-D-galactoside) analog.

In yet other embodiments, the present disclosure provides for kits for an assay format that defines the order of the reagent and sample additions for assaying a vitamin D moiety in a sample, which assay format comprises: a) a sample vitamin D is first contacted with an acidic pH buffer and a vitamin D binding partner (e.g., antibody or antibodies) before addition of the ED-25(OH)D conjugate to the reaction mixture; and b) the ED-25(OH)D conjugate and EA protein are added to the reaction mixture sequentially or in two separated steps.

In yet other embodiments, the present disclosure provides for a homogeneous assay format for assaying a vitamin D moiety in a sample. The assay format does not involve a phase separation step and does not remove any sample proteins out of the reaction mixture during the assay.

In some embodiments, an exemplary method comprises the steps of contacting a sample containing or suspected of containing a vitamin D moiety with an acidic buffer solution and a specific binding partner that specifically binds to said vitamin D moiety, if present in said sample, said binding partner being different from a natural vitamin D binding protein for said vitamin D moiety, and b) an ED-25(OH)D conjugate which is added into the reaction mixture only after the sample has been first contacted with an acidic buffer solution and a vitamin D binding partner (e.g., antibody or antibodies); and c) assessing the binding between said specific binding partner and said vitamin D moiety to determine the presence, absence and/or amount of said vitamin D moiety in said sample by addition of an enzyme acceptor (EA) and determination of β-galactosidase activity in the presence of a β-galactosidase substrate, e.g., a liquid stable β-galactosidase substrate.

In some embodiments, an exemplary method is conducted in a homogeneous assay format. The exemplary method provides for a specific reaction scheme that defines the sequence of additions of sample and reagents for assaying vitamin D in biological samples. The exemplary assay scheme requires that the sample be first contacted by an acidic buffer solution and a vitamin D binding partner (e.g., antibody or antibodies) before the sample is contacted by an ED-25(OH)D; and ED-25(OH) D and EA protein are added into the reaction mixture in a two separated steps.

In some embodiments, the present disclosure provides for vitamin D assay kits that are packaged in liquid stable format for all reagents included in the kits.

In one example, a sample is contacted with an acidic buffer solution and a specific binding partner (e.g., antibody or antibodies). For example, a sample can be contacted with an acidic buffer solution of pH values below 4.0, and a specific binding partner (e.g., antibody and antibodies) before being contacted with a ED-25(OH)D conjugate. The exemplary contact order can be: 1) an acidic pH buffer solution, a specific binding partner (e.g., antibody or antibodies); 2) after a period of incubation of the step (1), the sample is further contacted with an ED-25(OH)D conjugate; and 3) a EA protein is introduced into the reaction mixture for determination of α-complementation of the newly assembled β-galactosidase activity in the reaction mixture.

In another example, a sample can be contacted with a vitamin D binding partner (e.g., antibody or antibodies) prior to be contacted by an acidic pH buffer. The exemplary contact order can be: 1) sample is contacted with a vitamin D binding partner (e.g., antibody or antibodies) followed by contact with an acidic pH buffer solution; 2) after a period of incubation of the step (1), the sample is further contacted with an ED-25(OH)D conjugate; and 3) a EA protein is introduced into the reaction mixture for determination of α-complementation of the newly assembled β-galactosidase activity in the reaction mixture.

In still another example, a sample can be contacted with an acidic pH buffer solution prior to be contacted by a vitamin D binding partner (e.g., antibody or antibodies). The exemplary contact order can be: 1) sample is contacted with an acidic pH buffer solution followed by contact with vitamin D binding partner (antibody or antibodies); 2) after a period of incubation of the step (1), the sample is further contacted with an ED-25(OH)D conjugate; and 3) a EA protein is introduced into the reaction mixture for determination of α-complementation of the newly reassembled β-galactosidase activity in the reaction mixture.

In yet example, a sample can be contacted with an acidic pH buffer solution prior to be contacted by a vitamin D binding partner (e.g., antibody or antibodies). The exemplary contact order can be: 1) contact with an acidic pH buffer solution followed by contact with vitamin D binding partner (e.g., antibody or antibodies); 2) after a period of incubation of the step (1), the sample is further contacted with an EA protein followed by contact with an ED-25(OH)D conjugate.

In yet another example, a sample can be contacted with a vitamin D binding partner (e.g., antibody or antibodies) prior to be contacted by an acidic pH buffer. The exemplary contact order can be: 1) contact with a vitamin D binding partner (e.g., antibody or antibodies) followed by contact with an acidic pH buffer solution; 2) after a period of incubation of the step (1), the sample is further contacted with an EA protein followed by contact with ED-25(OH)D conjugate.

In some embodiments, the present methods do not comprise a step of contacting the sample with 8-anilino-1-naphthalenesulfonic acid ammonium salt and/or 3-(acetonylbenzyl)-4-hydroxycoumarin. See e.g., U.S. Pat. No. 7,482,162 B2. In other embodiments, the present methods do not comprise a step of contacting the sample with a non-competitive displacement agent that separates the vitamin D moiety from its binding protein in the sample. Id.

The pH in a final reaction mixture comprising the sample, the acidic pH buffer, the vitamin D binding partner (e.g., antibody or antibodies), the ED-25(OH)D conjugate, the EA protein and the β-galactosidase substrate can be at any suitable value or range. In some embodiments, the pH in a final reaction mixture comprising the sample, the acidic pH buffer, the vitamin D binding partner (e.g., antibody or antibodies), the ED-25(OH)D conjugate, the EA protein and the β-galactosidase substrate is at 5 or higher. In other embodiments, the pH in a final reaction mixture containing entire reaction components is at 10 or lower. In still other embodiments, the pH in a final reaction mixture comprising the sample, the acidic pH buffer solution, the vitamin D binding partner (e.g., antibody or antibodies), the ED-25(OH)D conjugate, the EA protein, the β-galactosidase substrate and stabilizers is in a range from about 4 to about 13, e.g., at about 4, 4.5, 5, 5.5, 5.6, 5.7, 5.8, 5.9, 6, 6.5, 7, 7.5, 8, 8.5, 9, 9.5, 10, 10.5, 11, 11.5, 12, 12.5, or 13.

In some embodiments, the present disclosure provides for a kit for assaying a vitamin D moiety in a sample, which kit comprises: a) an acidic pH buffer solution; b) a specific binding partner that specifically binds to said vitamin D moiety, if present in said sample, said binding partner being different from a natural vitamin D binding protein for said vitamin D moiety; and c) a ED-25(OH) D conjugate; and d) a EA protein and a substrate for β-galactosidase.

In some embodiments, the present disclosure provides for a kit for assaying a vitamin D moiety in a sample, which kit comprises: a) an acidic pH buffer solution; b) a specific binding partner that specifically binds to said vitamin D moiety, if present in said sample, said binding partner being different from a natural vitamin D binding protein for said vitamin D moiety; and c) a ED-25(OH) D conjugate; d) a EA protein and a substrate for β-galactosidase; e) and a set of vitamin D calibrator with known vitamin D values.

In some embodiments, the present invention provides a kit for assay a vitamin D moiety in a sample, which kit comprises; a) an acidic pH buffer solution containing a substrate for β-galactosidase; b) a specific binding partner that specifically binds to said vitamin D moiety, if present in said sample, said binding partner being different from a natural vitamin D binding protein for said vitamin D moiety; and c) a ED-25(OH) D conjugate; d) a EA protein; and e) a set of vitamin D calibrators.

In some embodiments, the present disclosure provides for a kit for assaying a vitamin D moiety in a sample, which kit comprises: a) an acidic pH buffer solution containing a substrate for β-galactosidase and a specific binding partner that specifically binds to said vitamin D moiety, if present in said sample, said binding partner being different from a natural vitamin D binding protein for said vitamin D moiety; and b) a ED-25(OH) D conjugate; c) a EA protein; and e) a set of vitamin D calibrators.

The reagents or components in the present kits can be formulated or arranged in any suitable fashion or form. In some embodiments, the present kits comprise the following reagents: (1) a sample dilution buffer containing a specific vitamin D binding partner (In some embodiments antibody or antibodies); (2) a first assay reagent (R1) comprising the acidic buffer solution and a β-galactosidase substrate; (3) a second assay reagent (R2) comprising a ED-25(OH)D conjugate; and (4) a third assay reagent (R3) comprising an EA protein.

The above kits can be used in a method for assaying a vitamin D moiety in a sample, which method comprises: a) forming a mixture of a sample, the first assay reagent and the second assay reagent and incubating the mixture for a period of time before adding the third assay reagent to the mixture; and b) quantifying the amount of 25(OH)D in the sample by measuring the optical change of the reaction mixture and using a set of 25(OH)D calibrators.

The present invention is further illustrated by the following exemplary embodiments.

1. A method for assaying a vitamin D moiety in a sample, which method comprises:

a) contacting a sample containing or suspected of containing a vitamin D moiety with
1) a buffer of acidic pH,
2) a specific binding partner that specifically binds to said vitamin D moiety, if present in said sample, to form a vitamin D moiety/specific binding partner complex, said binding partner being different from a natural vitamin D binding protein for said vitamin D moiety,
3) an enzyme donor (ED)-vitamin D moiety conjugate, said enzyme donor (ED) comprising a first fragment of a β-galactosidase, and
4) an enzyme acceptor (EA), said enzyme acceptor (EA) comprising a second fragment of a β-galactosidase,

wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase, said sample is contacted with said buffer of acidic pH and said specific binding partner in one or more steps, and said sample is contacted with said ED-vitamin D moiety conjugate and said enzyme acceptor (EA) in other separate one or more steps; and

b) assessing binding between said specific binding partner and said vitamin D moiety to determine the presence, absence and/or amount of said vitamin D moiety in said sample by measuring the activity of said reassembled active β-galactosidase in the presence of a β-galactosidase substrate.

2. The method of embodiment 1, which does not comprise a step of removing the natural vitamin D binding protein for the vitamin D moiety prior to assessing binding between the specific binding partner and the vitamin D moiety.

3. The method of embodiment 1 or 2, which does not comprise a wash step.

4. The method of any of embodiments 1-3, which is conducted as a homogeneous assay.

5. The method of any of embodiments 1-4, wherein the vitamin D moiety is vitamin D₃, vitamin D₂, a vitamin D metabolite, 1,25-dihydroxyvitamin D₃ (1,25-(OH)₂D₃), or 3-epi-25-hydroxyvitamin D3 (3-epi-25(OH)D3 or C3-epimer).

6. The method of embodiment 5, wherein the vitamin D metabolite is 25-hydroxy-vitamin D (25(OH)D).

7. The method of embodiment 6, wherein the 25(OH)D is 25(OH)D3.

8. The method of embodiment 6, wherein the 25(OH)D is 25(OH)D2.

9. The method of embodiment 6, wherein the 25(OH)D is a sum of 25(OH)D2 and 25(OH)D3.

10. The method of any of embodiments 1-9, wherein the buffer of acidic pH has a pH ranging from about 1.0 to about 5.0.

11. The method of embodiment 10, wherein the buffer of acidic pH has a pH ranging from about 2.0 to about 4.0.

12. The method of any of embodiments 1-11, wherein the specific binding partner that specifically binds to the vitamin D moiety is an antibody that specifically binds to the vitamin D moiety.

13. The method of embodiment 12, wherein the antibody specifically binds to 25(OH)D.

14. The method of embodiment 13, wherein the antibody specifically binds to 25(OH)D 2 and/or 25(OH)D 3.

15. The method of embodiment 14, wherein the antibody specifically binds to 25(OH)D 2 and 25(OH)D 3 with a similar binding affinity.

16. The method of any of embodiments 1-15, wherein the vitamin D moiety to be assayed and the vitamin D moiety in the ED-vitamin D moiety conjugate have the same or a similar affinity towards the vitamin D binding partner.

17. The method of embodiment 16, wherein the vitamin D moiety is vitamin D₃, vitamin D₂, a vitamin D metabolite or 1,25-dihydroxyvitamin D₃ (1,25-(OH)₂D₃).

18. The method of embodiment 17, wherein the vitamin D metabolite is 25-hydroxy-vitamin D (25(OH)D).

19. The method of embodiment 18, wherein the 25(OH)D is 25(OH)D 3.

20. The method of embodiment 18, wherein the 25(OH)D is 25(OH)D 2.

21. The method of any of embodiments 1-20, wherein the first fragment of a β-galactosidase in the enzyme donor (ED) and the second fragment of a β-galactosidase in the enzyme acceptor (EA) are prepared separately.

22. The method of any of embodiments 1-20, wherein the first fragment of a β-galactosidase in the enzyme donor (ED) and the second fragment of a β-galactosidase in the enzyme acceptor (EA) are prepared simultaneously.

23. The method of embodiment 22, wherein the β-galactosidase is E. coli wild type β-galactosidase or its mutants.

24. The method of embodiment 23, wherein the second fragment of a β-galactosidase in the enzyme acceptor (EA) comprises a deletion near the amino terminus of about 5%-10% of the β-galactosidase single subunit.

25. The method of embodiment 24, wherein the second fragment of a β-galactosidase in the enzyme acceptor (EA) comprises about 990 amino acid residues.

26. The method of embodiment 25, wherein the second fragment of a β-galactosidase in the enzyme acceptor (EA) is comprised within amino acid residues 20 and 1024 of E. coli β-galactosidase.

27. The method of embodiment 23, wherein the first fragment of a β-galactosidase in the enzyme donor (ED) comprises about 40 to 100 amino acid residues of the β-galactosidase that are missing from the second fragment of a β-galactosidase in the enzyme acceptor (EA).

28. The method of embodiment 27, wherein the first fragment of a β-galactosidase in the enzyme donor (ED) is comprised within amino acid residues 1 and 100 of E. coli β-galactosidase.

29. The method of embodiment 28, wherein the first fragment of a β-galactosidase in the enzyme donor (ED) is modified to contain a cysteine residue for covalent attachment to the vitamin D moiety.

30. The method of any of embodiments 1-29, wherein the sample is contacted with the buffer of acidic pH and the specific binding partner before the sample is contacted with the ED-vitamin D moiety conjugate and/or the enzyme acceptor (EA).

31. The method of embodiment 30, wherein the sample is first contacted by the vitamin D binding partner (e.g., antibody or antibodies) and the acidic pH buffer solution prior to be contacted by the ED-vitamin D moiety conjugate (e.g., ED-25(OH)D conjugate) and the enzyme acceptor (EA) simultaneously.

32. The method of embodiment 30, wherein the sample is first contacted by the vitamin D binding partner (e.g., antibody or antibodies) and the acidic pH buffer solution prior to be contacted by the ED-vitamin D moiety conjugate (e.g., ED-25(OH)D conjugate) and the enzyme acceptor (EA) sequentially.

33. The method of embodiment 32, wherein the sample is contacted with the vitamin D binding partner (e.g., antibody or antibodies) and the acidic pH buffer solution, then contacted with the ED-vitamin D moiety conjugate (e.g., ED-25(OH)D conjugate), and then contacted with the enzyme acceptor (EA).

34. The method of embodiment 30, wherein the additions of the sample and reagents follow a specific sequence or order in which:

1) the sample is first diluted with a buffer comprising a vitamin D binding partner (e.g., antibody or antibodies), and part of the diluted sample is then contacted with the acidic buffer solution comprising a β-galactosidase substrate prior to the additions of the ED-vitamin D moiety conjugate (e.g., ED-25(OH)D conjugate) and the enzyme acceptor (EA); and
2) the ED-vitamin D moiety conjugate (e.g., ED-25(OH)D conjugate) and the enzyme acceptor (EA) are added into the reaction mixture sequentially or in two separated steps.

35. The method of embodiment 30, wherein the additions of the sample and reagents follow a specific sequence or order in which:

1) the sample is first contacted by the acidic buffer solution comprising a specific Vitamin D binding partner (e.g., antibody or antibodies) and a β-galactosidase substrate prior to the additions of the ED-vitamin D moiety conjugate (e.g., ED-25(OH)D conjugate) and the enzyme acceptor (EA); and
2) the ED-vitamin D moiety conjugate (e.g., ED-25(OH)D conjugate) and the enzyme acceptor (EA) are added into the reaction mixture sequentially or in two separated steps.

36. The method of embodiment 30, which is a homogenous assay that is conducted in a single reaction container (e.g., a cuvette) comprising the steps of sample dilution with a buffer comprising the vitamin D binding partner, the acidic pH buffer solution comprising a β-galactosidase substrate, the ED-vitamin D moiety conjugate (e.g., ED-25(OH)D conjugate), the enzyme acceptor (EA) and one or more stabilizers.

37. The method of embodiment 36, wherein the one or more stabilizers is selected from the group consisting of a simple polyol (sugar alcohol) compound, e.g., glycerol, a sugar alcohol, e.g., sorbitol and a reducing agent, e.g., TCEP.

38. The method of embodiment 30, which is a homogenous assay that is conducted in two separated containers (e.g., two cuvettes) with the sample being first diluted with a buffer comprising the vitamin D binding partner in one cuvette (sample dilution cuvette) followed by mixing part of the diluted sample with the acidic pH buffer solution comprising a β-galactosidase substrate in another separated cuvette (reaction cuvette) before additions of the ED-vitamin D moiety conjugate (e.g., ED-25(OH)D conjugate), and the enzyme acceptor (EA).

39. The method of any of embodiments 1-38, wherein the β-galactosidase substrate is o-nitrophenyl-β-D-galactoside (ONPG), chlorophenol red-β-D-galactopyranoside (CPRG), or an analogue thereof.

40. The method of any of embodiments 1-39, which has a total assay time that is about 30 minutes or shorter.

41. The method of any of embodiments 1-40, which is conducted on a general chemistry analyzer or a clinical chemistry analyzer.

42. The method of embodiment 41, wherein the general chemistry analyzer or the clinical chemistry analyzer is capable of taking at least 3 reagents in an assay.

43. The method of embodiment 41 or 42, wherein the general chemistry analyzer or clinical chemistry analyzer is from Roche, Modular P, Cobas series, Hitachi series, Mindray BS series, Horiba Pentra, alpha Wassermann ACE system; and Siemens Dimension.

44. The method of any of embodiments 1-43, which is used to assess status of the vitamin D moiety in a subject, and the sample is a biological sample obtained and/or derived from the subject.

45. The method of embodiment 44, wherein the subject is a mammal.

46. The method of embodiment 45, wherein the mammal is a human.

47. The method of any of embodiments 44-46, wherein the sample is a biological fluid.

48. The method of embodiment 47, wherein the biological fluid is selected from the group consisting of whole blood, plasma, serum and urine.

49. A kit for assaying a vitamin D moiety in a sample, which kit comprises:

a) a buffer of acidic pH,

b) a specific binding partner that specifically binds to a vitamin D moiety, if present in said sample, to form a vitamin D moiety/specific binding partner complex, said binding partner being different from a natural vitamin D binding protein for said vitamin D moiety,

c) an enzyme donor (ED)-vitamin D moiety conjugate, said enzyme donor (ED) comprising a first fragment of a β-galactosidase, and

d) an enzyme acceptor (EA), said enzyme acceptor (EA) comprising a second fragment of a β-galactosidase,

wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

50. The kit of embodiment 49, which further comprises means for assessing binding between the specific binding partner and the vitamin D moiety to determine the presence, absence and/or amount of the vitamin D moiety in the sample.

51. The kit of embodiment 50, wherein the means for assessing binding between the specific binding partner and the vitamin D moiety comprises a β-galactosidase substrate or a vitamin D calibrator.

52. The kit of embodiment 51, wherein the β-galactosidase substrate is comprised in the buffer of acidic pH.

53. The kit of embodiment 51, which comprises a set of vitamin D calibrators of known vitamin D values.

54. The kit of embodiment 49, which comprises reagents:

a) a sample dilution buffer comprising the specific vitamin D binding partner (e.g., antibody or antibodies);

b) a first assay reagent (R1) comprising a β-galactosidase substrate in the buffer of acidic pH;
c) a second assay reagent (R2) comprising the ED-vitamin D moiety conjugate (e.g., ED-25(OH)D conjugate); and
d) a third assay reagent (R3) comprising the enzyme acceptor (EA).

55. The kit of embodiment 54, which comprises the sample dilution buffer, R1, R2 and R3 in a liquid stable format.

56. The kit of embodiment 54 or 55, which further comprises a vitamin D calibrator.

57. The kit of embodiment 56, which comprises a set of vitamin D calibrators of known vitamin D values.

58. A kit for assaying a vitamin D moiety in a sample, which kit comprises:

a) a first assay reagent (R1) comprising an acidic pH buffer solution comprising a β-galactosidase substrate and a specific binding partner that specifically binds to said vitamin D moiety, if present in said sample, said binding partner being different from a natural vitamin D binding protein for said vitamin D moiety;

b) a second assay reagent (R2) comprising an enzyme donor (ED)-vitamin D moiety conjugate, said enzyme donor (ED) comprising a first fragment of a β-galactosidase; and

c) a third assay reagent (R3) comprising an enzyme acceptor (EA), said enzyme acceptor (EA) comprising a second fragment of a β-galactosidase,

wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

59. The kit of embodiment 58, which comprises the R1, R2 and R3 in a liquid stable format or in a solid format, e.g., lyophilized format, or in a mixed format of a liquid and solid e.g., lyophilized.

60. A method for assaying a vitamin D moiety in a sample using a kit of any of embodiments 54-57, which method comprises:

a) diluting a sample with the sample dilution buffer comprising the specific vitamin D binding partner, mixing part of the diluted sample with R1, incubating the mixture for a period of time, adding R2, and after another period of incubation time, adding R3 to the reaction mixture; and

b) quantifying the amount of a vitamin D moiety (e.g., 25(OH)D) in the sample by measuring the optical change of the reaction mixture and using a set of vitamin D calibrators of known vitamin D values (e.g., a set of 25(OH)D calibrators).

61. A method for assaying a vitamin D moiety in a sample using the kit of embodiment 58 or 59, which method comprises;

a) mixing a sample with R1 to form a reaction mixture;

b) after a period of incubation, adding R2 and R3 to the reaction mixture in two separate steps;

c) quantifying the amount of a vitamin D moiety (e.g., 25(OH)D) in the sample by measuring the optical change of the reaction mixture and using a set of vitamin D calibrators of known vitamin D values (e.g., a set of 25(OH)D calibrators).

62. A reaction mixture for assaying a vitamin D moiety in a sample, which reaction mixture comprises, in an acidic environment:

a) a specific binding partner that specifically binds to a vitamin D moiety, if present in said sample, to form a vitamin D moiety/specific binding partner complex, said binding partner being different from a natural vitamin D binding protein for said vitamin D moiety,

b) an enzyme donor (ED)-vitamin D moiety conjugate, said enzyme donor (ED) comprising a first fragment of a β-galactosidase, and

c) an enzyme acceptor (EA), said enzyme acceptor (EA) comprising a second fragment of a β-galactosidase,

wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

63. The reaction mixture of embodiment 62, which is in a single phase.

64. A method for assaying a vitamin D moiety in a sample, which method utilizes the cloned enzyme donor immunoassay (CEDIA) principle and comprises:

a) contacting a sample containing or suspected of containing a vitamin D moiety with a buffer of acidic pH and a specific binding partner that specifically binds to said vitamin D moiety, if present in said sample, said binding partner being different from a natural vitamin D binding protein for said vitamin D moiety, and
b) adding the enzyme donor (ED)-25(OH)D conjugate and the enzyme acceptor (EA) protein to the reaction mixture of the step (a) in a two separated steps, and
c) assessing binding between said specific binding partner and said vitamin D moiety to determine the presence, absence and/or amount of said vitamin D moiety in said sample by measuring the activity of β-galactosidase of the reaction mixture in the presence of a β-galactosidase substrate.

65. The method of embodiment 64, which does not comprise a step of removing the natural vitamin D binding protein for the vitamin D moiety or does not comprise a wash step in the entire assay process.

66. The method of embodiment 64 or 65, which is conducted in a homogeneous assay format.

67. The method of any of embodiments 64-66, wherein the sample is first contacted with an acidic buffer and a vitamin D binding partner, preferably a specific antibody or antibodies against 25(OH) vitamin D, before the sample is contacted by the ED-25(OH)D conjugate.

68. The method of any of embodiments 64-67, wherein the acidic buffer is of a pH ranging from 1.0 to 5.0, preferably ranging from pH 2.0 to 4.0.

69. The method of any of embodiments 64-68, wherein the vitamin D binding partner is an antibody having a specific binding affinity towards 25(OH) vitamin D including 25(OH) vitamin D3 and 25(OH) vitamin D2, preferably with a similar binding affinity towards vitamin D3 and D2 molecules.

70. The method of embodiment 69, wherein the vitamin D moiety is 25(OH) vitamin D2, 25(OH) vitamin D3 or a sum of 25(OH) vitamin D2 and 25(OH) vitamin D3.

71. The method of any of embodiments 64-70, wherein the additions of the sample and reagents follow a specific sequence or order in which: 1) the sample is first contacted by a vitamin D binding partner (e.g., antibody or antibodies) and an acidic pH buffer solution prior to be contacted by the ED-25(OH)D conjugate and the EA protein; and 2) ED-25(OH)D conjugate and the EA protein are added to the reaction mixture sequentially or in two separated steps.

72. The method of any of embodiments 64-70, wherein the additions of the sample and reagents follow a specific sequence or order in which: 1) the sample is first contacted by an acidic pH buffer solution and a vitamin D binding partner (e.g., antibody or antibodies) prior to be contacted by the ED-25(OH)D conjugate and the EA protein; and 2) ED-25(OH)D conjugate and EA protein are added into the reaction mixture sequentially or in two separated steps.

73. The method of any of embodiments 64-70, wherein the additions of the sample and reagents follow a specific sequence or order in which: 1) the sample is first diluted with a buffer containing a vitamin D binding partner (e.g., antibody or antibodies), and part of the diluted sample is then contacted with an acidic buffer solution containing a β-galactosidase substrate prior to the additions of the ED-25(OH)D conjugate and the EA protein; and 2) ED-25(OH)D conjugate and EA protein are added into the reaction mixture sequentially or in two separated steps.

74. The method of any of embodiments 64-70, wherein the additions of the sample and reagents follow a specific sequence or order in which: 1) the sample is first contacted by an acidic buffer solution containing a specific Vitamin D binding partner (e.g., antibody or antibodies) and a β-galactosidase substrate prior to the additions of the ED-25(OH)D conjugate and the EA protein; and 2) ED-25(OH)D conjugate and EA protein are added into the reaction mixture sequentially or in two separated steps.

75. The method of any of embodiments 64-74, which is a homogenous assay that is conducted in a single reaction container, e.g., a cuvette, including the steps of sample dilution with a buffer containing the vitamin D binding partner, the acidic pH buffer solution containing a β-galactosidase substrate, the ED-25(OH)D conjugate, the EA protein and the stabilizers.

76. The method of any of embodiments 64-74, which is a homogenous assay that is conducted in two separated container, e.g., cuvettes, with the sample being first diluted with a buffer containing the vitamin D binding partner in a container, e.g., cuvette, (sample dilution container) followed by mixing part of the diluted sample with an acidic pH buffer solution containing the β-galactosidase substrate in a separated container, e.g., cuvette, (reaction container) before additions of the ED-25(OH)D conjugate and the EA protein.

77. The method of any of embodiments 64-76, which has a total assay time that is about 30 minutes or shorter. In some embodiments, the total assay time includes the time needed for pipetting samples and reagents, and the time needed for incubations in each step of the assay.

78. The method of any of embodiments 64-77, which is conducted on a general chemistry analyzer or a clinical chemistry analyzer, especially those analyzers that are capable of taking 3 reagents in an assay.

79. A kit for assaying a vitamin D moiety in a sample, which kit comprises:

a) an acidic pH buffer solution containing a β-galactosidase substrate;

b) a specific binding partner that specifically binds to said vitamin D moiety, if present in said sample, said binding partner being different from a natural vitamin D binding protein for said vitamin D moiety;

c) an enzyme donor (ED)-25(OH)D conjugate; and

d) an enzyme acceptor (EA) protein.

80. The kit of embodiment 79, which further comprises means for assessing binding between the specific binding partner and the vitamin D moiety to determine the presence, absence and/or amount of the vitamin D moiety in the sample to include a set of vitamin D calibrators of known vitamin D values.

81. The kit of embodiment 79 or 80, which comprises reagents:

(1) a sample dilution buffer containing a specific vitamin D binding partner (e.g., an antibody);

(2) a first assay reagent (R1) comprising a β-galactosidase substrate in a buffer of acidic pH;

(3) a second assay reagent (R2) comprising the enzyme donor (ED)-25(OH)D conjugate; and
(4) a third assay reagent (R3) comprising the enzyme acceptor (EA) protein.

82. The kit of embodiment 81, which comprises 3 assay reagents and one sample dilution buffer that are all in a liquid stable format.

83. The kit of any of embodiments 79-82, which further includes a set of vitamin D calibrators.

84. A method for assaying a vitamin D moiety in a sample using the kit of embodiment 83, which method comprises:

a) diluting a sample with a sample dilution buffer containing a vitamin D binding partner, mixing part of the diluted sample with the first assay reagent, incubating the mixture for a period of time, adding the second assay reagent, and after another period of incubation time, adding the third assay reagent to the reaction mixture; and
b) quantifying the amount of 25(OH)D in the sample by measuring the optical change of the reaction mixture and using a set of 25(OH)D calibrators.

85. A kit for assaying a vitamin D moiety in a sample, which kit comprises:

a) first assay reagent (R1): an acidic pH buffer solution containing a β-galactosidase substrate and a specific binding partner that specifically binds to said vitamin D moiety, if present in said sample, said binding partner being different from a natural vitamin D binding protein for said vitamin D moiety;

b) a second assay reagent (R2): an enzyme donor (ED)-25(OH)D conjugate; and

c) a third assay reagent (R3): an enzyme acceptor (EA) protein.

86. The kit of embodiment 85, wherein R1, R2 and R3 are in liquid stable format, in lyophilized format, or in a mixed formats.

87. A method for assaying a vitamin D moiety in a sample using the kit of embodiment 85 or 86, which method comprises;

a) mixing the sample with the first assay reagent R1;

b) after a short period of incubation, adding the ED-25(OH)D and the EA protein to the reaction mixture in two separate steps; and

c) quantifying the amount of 25(OH)D in the sample by measuring the optical change of the reaction mixture and using a set of 25(OH)D calibrators.

### F. EXAMPLES

### Example 1

### Total 25(OH)D Assay Kit (Three Reagents+a Diluent Format)

One diluent and the Three Reagents used in this example are listed below:

### Diluent:

PBS: 0.5×

BSA: 0.3%

Sodium Azide: 0.1%

25-OH Vitamin D antibody: 20 nM

Final pH: 7.4

### Reagent R1:

Sodium Acetate: 50 mM

DMSO: 10%

Tween 20: 0.02%

O-NPG analogue: 5 mg/mL

Final pH: 3.0

### Reagent R2:

Sodium Phosphate: 100 mM

EGTA: 2 mM

Sodium Azide: 0.1%

Pefabloc: 0.1 mM

ED-25(OH)D conjugate: 10 nM

Final pH: 7.4

### Reagent R3:

Tris-HCl: 70 mM

Sodium Azide: 0.1%

Magnesium Chloride: 60 mM

Sodium Chloride: 50 mM

Glycerol: 11.1%

TCEP: 8.5 mM

EA protein: 3.0 mg/mL

Final pH: 7.4

### Assay Parameters for Roche Modular P (Two Cuvettes Format)

Assay parameters for Roche Modular P (two cuvettes format) used in this example are listed below:

Sample dilution: Original sample volume: 20.0 uL, Diluent volume: 155;

Reagent Volume (μL);

R1 75;

R2 150;

R3 75;

Diluted Sample volume 20 uL;

First reading cycle: 42 (˜756 s);

Last reading cycle: 63 (˜1134 s);

Wavelength: Primary 415 nm or 405 nm; secondary 600 nm; and

Calibration mode: Logit/Log 3P

Precision test results with the above application parameters are summarized in Table 1 below.

TABLE 1

| | | | Within- | Within- | Total | Total |
|---|---|---|---|---|---|---|
| | | Mean | run SD | run | SD | CV |
| Specimen | n | (ng/mL) | (ng/mL) | CV (%) | (ng/mL) | (%) |
| Control #1 | 20 | 23.1 | 1.47 | 6.4 | 1.68 | 7.3 |
| Control #2 | 20 | 45.7 | 2.06 | 4.5 | 2.12 | 4.6 |
| Sample #1 | 20 | 22.6 | 1.19 | 5.3 | 1.45 | 6.4 |
| Sample #2 | 20 | 31.7 | 1.42 | 4.5 | 1.81 | 5.7 |
| Sample #3 | 20 | 40.6 | 1.42 | 3.5 | 1.66 | 4.1 |
| Sample #4 | 20 | 48.6 | 2.32 | 4.8 | 2.41 | 4.9 |
| Sample #5 | 20 | 55.8 | 2.14 | 3.8 | 2.34 | 4.2 |
| Sample #6 | 20 | 65.4 | 2.03 | 3.1 | 2.42 | 3.7 |
| Sample #7 | 20 | 69.7 | 2.02 | 2.9 | 2.55 | 3.7 |
| Sample #8 | 20 | 92.8 | 2.52 | 2.7 | 3.40 | 3.7 |
| Sample #9 | 20 | 134.6 | 2.97 | 2.2 | 3.87 | 2.9 |
| Very low Sample #1 | 20 | 9.4 | 1.22 | 13.0 | 1.31 | 14.0 |
| Very low Sample #2 | 20 | 11.2 | 1.58 | 14.2 | 1.55 | 13.9 |

### Assay Parameters for Horiba Pentra 400 (Two Cuvettes Format)

Assay parameters for Horiba Pentra 400 (two cuvettes format) used in this example are listed below:

Sample dilution: dilution ratio 8.7 (31 uL sample+269 uL diluent);

Reagent Volume (μL);

R1 95.0

R2 190.0

R3 95.0

Diluted Sample volume 25 uL;

Wavelength: Primary 405 nm; Secondary: None;

First reading cycle: 52 (624 s);

Last reading cycle: 99 (1188 s); and

Calibration mode: Logit/Log 4.

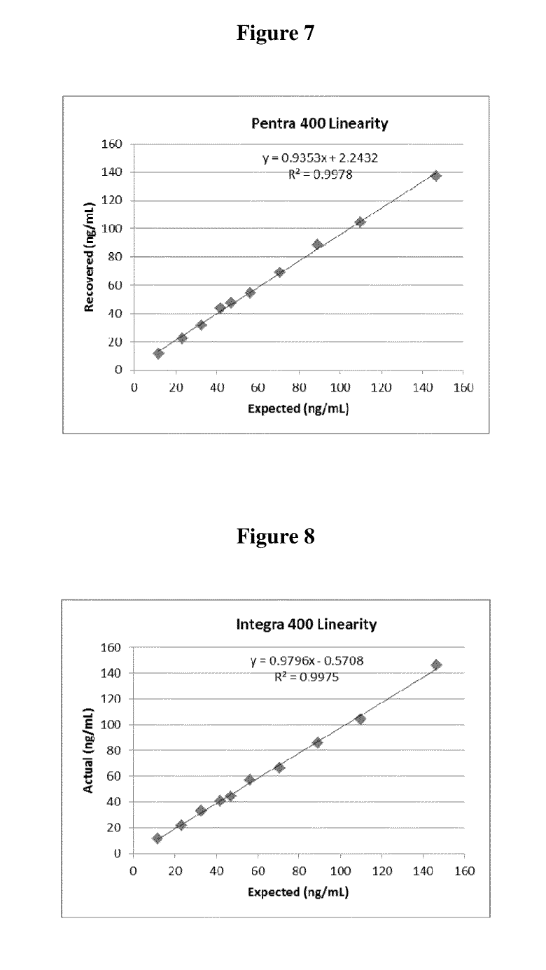

Linearity result obtained from Pentra 400 using the above application parameters is shown in FIG. 7.

### Assay Parameters for Roche Integra 400 (One Cuvette Format)

Assay parameters for Roche Integra 400 (one cuvette format) used in this example are listed below:

Sample dilution: sample volume: 3.0 uL, Diluent volume: 18 uL;

Reagent Volume (μL):

R1 55

R2 110

R3 55

First reading cycle: 91 (˜964 s);

Last reading cycle: 134 (˜1420 s);

Wavelength: Primary 409 nm; secondary 629 nm; and

Calibration mode: Logit/Log 4.

The above reaction was completed in a single cuvette. The linearity result obtained from Integra 400 using the above application parameters is shown in FIG. 8.

### Example 2

### Total 25(OH)D Assay Kit (Three Reagents)

The Three Reagents used in this example are listed below:

### Reagent R1:

Sodium Acetate: 50 mM

DMSO: 6%

Tween 20: 0.02%

BSA: 0.1%

25-OH Vitamin D antibody: 11.4 nM

O-NPG analogue: 5 mg/mL

Final pH: 3.0

### Reagent R2:

Sodium Phosphate: 100 mM

EGTA: 2 mM

Sodium Azide: 0.1%

Pefabloc: 0.1 mM

ED-25(OH)D conjugate: 10 nM

Final pH: 7.4

### Reagent R3:

Tris-HCl: 70 mM

Sodium Azide: 0.1%

Magnesium Chloride: 60 mM

Sodium Chloride: 50 mM

Glycerol: 11.1%

TCEP: 8.5 mM

EA protein: 3.0 mg/mL

Final pH: 7.4

### Assay Parameters for Roche Modular P (One Cuvette Format)

Assay parameters for Roche Modular P (one cuvette format) used in this example are listed below:

Sample volume: 3.0 μL;

Reagent Volume (μL);

R1 95

R2 160

R3 75

First reading cycle: 42 (˜756 s);

Last reading cycle: 63 (˜1134 s);

Wavelength: Primary 415 nm or 405 nm; secondary 600 nm; and

Calibration mode: Logit/Log 3P/

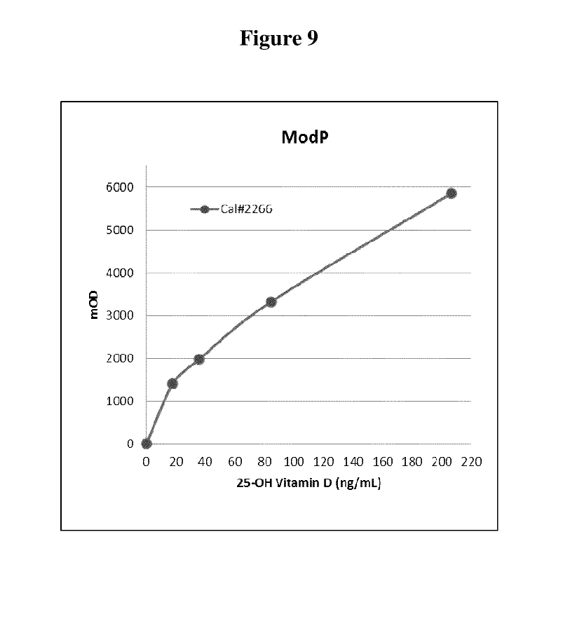

Calibration curve obtained from Roche Modular P using the above reagents and application parameters is depicted in FIG. 9.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for assaying a vitamin D moiety in a sample, which method comprises:
a) contacting a sample containing or suspected of containing a vitamin D moiety with
1) a buffer of acidic pH,
2) a specific binding partner that specifically binds to said vitamin D moiety, if present in said sample, to form a vitamin D moiety/specific binding partner complex, said binding partner being different from a natural vitamin D binding protein for said vitamin D moiety,
3) an enzyme donor (ED)-vitamin D moiety conjugate, said enzyme donor (ED) comprising a first fragment of a β-galactosidase, and
4) an enzyme acceptor (EA), said enzyme acceptor (EA) comprising a second fragment of a β-galactosidase,
wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase, said sample is contacted with said buffer of acidic pH and said specific binding partner in one or more steps, and said sample is contacted with said ED-vitamin D moiety conjugate and said enzyme acceptor (EA) in other separate one or more steps; and
b) assessing binding between said specific binding partner and said vitamin D moiety to determine the presence, absence and/or amount of said vitamin D moiety in said sample by measuring the activity of said reassembled active β-galactosidase in the presence of a β-galactosidase substrate.

2. The method of claim 1, wherein the vitamin D moiety is vitamin D₃, vitamin D₂, a vitamin D metabolite, 1,25-dihydroxyvitamin D₃ (1,25-(OH)₂D₃), or 3-epi-25-hydroxyvitamin D3 (3-epi-25(OH)D3 or C3-epimer).

3. The method of claim 1, wherein the buffer of acidic pH has a pH ranging from about 1.0 to about 5.0.

4. The method of claim 1, wherein the specific binding partner that specifically binds to the vitamin D moiety is an antibody that specifically binds to the vitamin D moiety.

5. The method of claim 1, wherein the vitamin D moiety to be assayed and the vitamin D moiety in the ED-vitamin D moiety conjugate have the same or a similar affinity towards the vitamin D binding partner.

6. The method of claim 1, wherein the β-galactosidase is E. coli wild type β-galactosidase or its mutants.

7. The method of claim 6, wherein the second fragment of a β-galactosidase in the enzyme acceptor (EA) comprises a deletion near the amino terminus of about 5%-10% of the β-galactosidase single subunit.

8. The method of claim 6, wherein the first fragment of a β-galactosidase in the enzyme donor (ED) comprises about 40 to 100 amino acid residues of the β-galactosidase that are missing from the second fragment of a β-galactosidase in the enzyme acceptor (EA).

9. The method of claim 1, wherein the sample is contacted with the buffer of acidic pH and the specific binding partner before the sample is contacted with the ED-vitamin D moiety conjugate and/or the enzyme acceptor (EA).

10. The method of claim 9, wherein the sample is contacted with the vitamin D binding partner (e.g., antibody or antibodies) and the acidic pH buffer solution, then contacted with the ED-vitamin D moiety conjugate (e.g., ED-25(OH)D conjugate), and then contacted with the enzyme acceptor (EA).

11. The method of claim 9, wherein the additions of the sample and reagents follow a specific sequence or order in which:
1) the sample is first diluted with a buffer comprising a vitamin D binding partner (e.g., antibody or antibodies), and part of the diluted sample is then contacted with the acidic buffer solution comprising a β-galactosidase substrate prior to the additions of the ED-vitamin D moiety conjugate (e.g., ED-25(OH)D conjugate) and the enzyme acceptor (EA); and
2) the ED-vitamin D moiety conjugate (e.g., ED-25(OH)D conjugate) and the enzyme acceptor (EA) are added into the reaction mixture sequentially or in two separated steps.

12. The method of claim 9, wherein the additions of the sample and reagents follow a specific sequence or order in which:
1) the sample is first contacted by the acidic buffer solution comprising a specific Vitamin D binding partner (e.g., antibody or antibodies) and a β-galactosidase substrate prior to the additions of the ED-vitamin D moiety conjugate (e.g., ED-25(OH)D conjugate) and the enzyme acceptor (EA); and
2) the ED-vitamin D moiety conjugate (e.g., ED-25(OH)D conjugate) and the enzyme acceptor (EA) are added into the reaction mixture sequentially or in two separated steps.

13. The method of claim 9, which is a homogenous assay that is conducted in a single reaction container (e.g., a cuvette) comprising the steps of sample dilution with a buffer comprising the vitamin D binding partner, the acidic pH buffer solution comprising a β-galactosidase substrate, the ED-vitamin D moiety conjugate (e.g., ED-25(OH)D conjugate), the enzyme acceptor (EA) and one or more stabilizers.

14. The method of claim 9, which is a homogenous assay that is conducted in two separated containers (e.g., two cuvettes) with the sample being first diluted with a buffer comprising the vitamin D binding partner in one cuvette (sample dilution cuvette) followed by mixing part of the diluted sample with the acidic pH buffer solution comprising a β-galactosidase substrate in another separated cuvette (reaction cuvette) before additions of the ED-vitamin D moiety conjugate (e.g., ED-25(OH)D conjugate), and the enzyme acceptor (EA).

15. The method of claim 1, wherein the β-galactosidase substrate is o-nitrophenyl-β-D-galactoside (ONPG), chlorophenol red-β-D-galactopyranoside (CPRG), or an analogue thereof.

16. The method of claim 1, which has a total assay time that is about 30 minutes or shorter.

17. The method of claim 1, which is conducted on a general chemistry analyzer or a clinical chemistry analyzer.

18. The method of claim 17, wherein the general chemistry analyzer or the clinical chemistry analyzer is capable of taking at least 3 reagents in an assay.

19. The method of claim 1, which is used to assess status of the vitamin D moiety in a subject, and the sample is a biological sample obtained and/or derived from the subject.

20. A kit for assaying a vitamin D moiety in a sample, which kit comprises:
a) a buffer of acidic pH,
b) a specific binding partner that specifically binds to a vitamin D moiety, if present in said sample, to form a vitamin D moiety/specific binding partner complex, said binding partner being different from a natural vitamin D binding protein for said vitamin D moiety,
c) an enzyme donor (ED)-vitamin D moiety conjugate, said enzyme donor (ED) comprising a first fragment of a β-galactosidase, and
d) an enzyme acceptor (EA), said enzyme acceptor (EA) comprising a second fragment of a β-galactosidase,
wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

21. The kit of claim 20, which further comprises means for assessing binding between the specific binding partner and the vitamin D moiety to determine the presence, absence and/or amount of the vitamin D moiety in the sample.

22. The kit of claim 21, wherein the means for assessing binding between the specific binding partner and the vitamin D moiety comprises a β-galactosidase substrate or a vitamin D calibrator.

23. The kit of claim 20, which comprises reagents:
a) a sample dilution buffer comprising the specific vitamin D binding partner (e.g., antibody or antibodies);
b) a first assay reagent (R1) comprising a β-galactosidase substrate in the buffer of acidic pH;
c) a second assay reagent (R2) comprising the ED-vitamin D moiety conjugate (e.g., ED-25(OH)D conjugate); and
d) a third assay reagent (R3) comprising the enzyme acceptor (EA).

24. The kit of claim 23, which comprises the sample dilution buffer, R1, R2 and R3 in a liquid stable format.

25. The kit of claim 24, which further comprises a vitamin D calibrator.

26. A kit for assaying a vitamin D moiety in a sample, which kit comprises:
a) a first assay reagent (R1) comprising an acidic pH buffer solution comprising a β-galactosidase substrate and a specific binding partner that specifically binds to said vitamin D moiety, if present in said sample, said binding partner being different from a natural vitamin D binding protein for said vitamin D moiety;
b) a second assay reagent (R2) comprising an enzyme donor (ED)-vitamin D moiety conjugate, said enzyme donor (ED) comprising a first fragment of a β-galactosidase; and
c) a third assay reagent (R3) comprising an enzyme acceptor (EA), said enzyme acceptor (EA) comprising a second fragment of a β-galactosidase,
wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.

27. The kit of claim 26, which comprises the R1, R2 and R3 in a liquid stable format or in a solid format, e.g., lyophilized format, or in a mixed format of a liquid and solid e.g., lyophilized.

28. A method for assaying a vitamin D moiety in a sample using a kit of claim 23, which method comprises:
a) diluting a sample with the sample dilution buffer comprising the specific vitamin D binding partner, mixing part of the diluted sample with R1, incubating the mixture for a period of time, adding R2, and after another period of incubation time, adding R3 to the reaction mixture; and
b) quantifying the amount of a vitamin D moiety (e.g., 25(OH)D) in the sample by measuring the optical change of the reaction mixture and using a set of vitamin D calibrators of known vitamin D values (e.g., a set of 25(OH)D calibrators).

29. A method for assaying a vitamin D moiety in a sample using the kit of claim 26, which method comprises;
a) mixing a sample with R1 to form a reaction mixture;
b) after a period of incubation, adding R2 and R3 to the reaction mixture in two separate steps;
c) quantifying the amount of a vitamin D moiety (e.g., 25(OH)D) in the sample by measuring the optical change of the reaction mixture and using a set of vitamin D calibrators of known vitamin D values (e.g., a set of 25(OH)D calibrators).

30. A reaction mixture for assaying a vitamin D moiety in a sample, which reaction mixture comprises, in an acidic environment:
a) a specific binding partner that specifically binds to a vitamin D moiety, if present in said sample, to form a vitamin D moiety/specific binding partner complex, said binding partner being different from a natural vitamin D binding protein for said vitamin D moiety,
b) an enzyme donor (ED)-vitamin D moiety conjugate, said enzyme donor (ED) comprising a first fragment of a β-galactosidase, and
c) an enzyme acceptor (EA), said enzyme acceptor (EA) comprising a second fragment of a β-galactosidase,
wherein when said ED-vitamin D moiety conjugate is not bound to said specific binding partner, said first fragment of a β-galactosidase in said enzyme donor (ED) and said second fragment of a β-galactosidase in said enzyme acceptor (EA) are configured to reassemble to form an active β-galactosidase.
